(19) United States
(12) Patent Application Publication
(10) Pub. No.: US 2003/0138476 A1
(43) Pub. Date: Jul. 24, 2003

(54) Use of glutamate and/or a glutamate precursor for the preparation of a nutritional or pharmaceutical preparation for the treatment or prevention of hyperpermeability or undesired permeability of the intestinal wall

(76) Inventors: Paulus Aluisius Marie Van Leeuwen, Amstelveen (NL); Alexander Petrus Johannes Houdijk, Amstelveen (NL); Cornelis Glas, Tietjerk (NL)

Correspondence Address: FITCH EVEN TABIN AND FLANNERY, 120 SOUTH LA SALLE STREET, SUITE 1600, CHICAGO, IL, 60603-3406, US

(21) Appl. No.: 10/203,789

(22) PCT Filed: Feb. 14, 2001
(86) PCT No.: PCT/NL01/00104

(30) Foreign Application Priority Data
Feb. 14, 2000 (NL) 1014380

(51) Int. Cl. A61K 35/50; A61K 47/00

(52) U.S. Cl. 424/439; 424/528

(57) ABSTRACT

The present invention relates to the use of glutamic acid for the preparation of a nutritional preparation that is intended for use for the treatment or prevention of excess or undesired permeability of the intestinal wall. In particular, according to the invention the glutamic acid is used in a nutritional preparation, such as a baby food or an enteral food. Examples of conditions where glutamic acid is used are: food allergy, internal drug allergy, sepsis, low blood flow through the intestines, ICU patients, surgical interventions, malnutrition or intestinal maturation of newborn babies.

The invention relates to the preparation of a nutritional preparation that is suitable for use in the case of conditions associated with an increased permeability of the intestinal wall.

The intestinal epithelium acts as a selective barrier which allows the absorption of nutrients but restricts the passage of microorganisms and undesired macromolecules. Maintaining this barrier is considered to be important in order to protect the host against the migration of pathogenic microorganisms from the intestines to the bloodstream. It is assumed that the increase in the permeability of the intestines is associated with damage to the paracellular transport system of the intestinal mucosa, as a result of which translocation of endotoxins and (pathogenic) bacteria can occur. As a result of the damage to the intestinal mucosa it is also possible for absorption of macromolecules to occur, which are then able to initiate allergic reactions.

An increase in the permeability of the intestinal wall has been detected in clinical conditions associated with damage to the intestinal mucosa barrier, such as endotoxaemia, sepsis, multiple trauma, malnutrition, major surgical interventions, parenteral nutrition and burns. An increase in the permeability to larger molecules, such as proteins, has been found in the newborn, but also occurs in healthy people if they are allergic to food products.

It is known that glutamine is able to lower the macromolecular hyperpermeability of intestinal cells which is induced by phorbol 12,13-dibutyrate (Kouznetsova et al. J. Parenteral Enteral Nutrition, 23 (1999) 136-139). A disadvantage of glutamine, however, is that it is not stable at room temperature, which renders it unsuitable for (non-chilled) foods with a long shelf life. Moreover, glutamine has poor solubility.

Instead of glutamine, specific products based on peptides, mainly di- and tripeptides, are used. These peptides are frequently prepared from glutamine-rich vegetable proteins, such as, in particular, wheat protein, in which preparation method, following enzymatic conversion, fractionation technology is used in order to obtain the specific peptide fraction as the main fraction. Examples thereof can be found in JP 05236909 and JP 08157385. Such a preparation of these peptides is expensive and complex. Moreover, a product obtained from wheat protein can be problematic for some patients, such as coeliac patients.

It has now been found that too high a permeability of the intestinal wall can be effectively treated or prevented by the administration of a suitable quantity of glutamate and/or a glutamate precursor, preferably in a nutritional preparation. The invention therefore relates to a nutritional or pharmaceutical preparation containing glutamate and/or a glutamate precursor, in particular for such a use, as described in more detail in the appended claims.

From the state of the art. for instance U.S. Pat. No. 5,366,723 it is known to use a combination of glutamic acid, aspartic acid and cystein in decreasing the toxicity of platinum compounds in the treatment of cancer. One of the activities mentioned is regeneration of the intestinal mucosa. However, regeneration of the intestinal mucosa relates to a different phenomena than permeability of the intestine which is in particular associated with integrity of the intestine or the paracellular transport via the intestinal mucosa.

Here a nutritional preparation is understood to be a composition that contains food constituents (at least one), such as proteins, carbohydrates, fats, vitamins, minerals and the like. Preferably the composition contains more than one food constituent and preferably it contains all necessary food constituents. It can therefore be a food supplement or a complete food or a food drug (“neutraceuticum”).

The nutritional preparation of the invention can be an infant formula or children's food or an enteral (functional/clinical/problem solving) food.

Substances that are known to have a beneficial effect on the intestinal function (permeability) can also be added. In particular, one or more polyamines such as spermine, spermadine or putrescine or one or more polyamine precursors, in particular ornithin and arginin can be used. Such polyamines are, for example, described in Dorhout et al., Br. J. Nutrition, 1997, 639-654 and in a report of a seminar held on Jun. 29to Jul. 2, 1999 in Glasgow, published in Proceedings of the Nutrition Society, Vol. 59 (Issue 1), 2000, 81-86. Polyamines or the precursors thereof can have a beneficial contribution in e.g. postnatal intestinal maturation, permeability of the intestine to macromolecules (allergy), the translocation of bacteria, etc.

The preparation contains preferably 0.05 to 10 g, more preferably 0.2 to 4 g free glutamate per 100 g of the nutritional preparation (dry weight). The polyamines are preferably present in an amount of 1 to 10 mg per 100 g of the nutritional preparation (dry weight).

The glutamate is preferably incorporated in a nutritional preparation alongside proteins or peptides, such as lactic or vegetable proteins. The nutritional preparation contains in particular lactic proteins or hydrolysates obtained therefrom. Lactic proteins comprise casein, whey proteins and lactoferrin.

Carbohydrates are understood to be digestible carbohydrates, such as glucose, lactose, maltose and sucrose, and digestible oligosaccharides and polysaccharides, such as maltodextrins, amylopectins and starch, as well as non-digestible carbohydrates (food fibres) such as galacto-oligosaccharides or fructo-oligosaccharides (inulin), vegetable and animal and microbial gums, such as carob bean flour and gum arabic. Fats comprise vegetable and animal fats, fats with medium length chains (C₈-C₁₂) (MCT), fats with unsaturated long chains (such as γ-linolenic acid, arachidonic acid, eicosapentaenoic acid and docosahexaenic acid).

The nutritional preparation according to the invention can also contain glutamine or an equivalent thereof. Glutamine equivalents are known to those skilled in the art. Examples of these are the abovementioned dipeptides and tripeptides. If the nutritional preparation is a food for babies or toddlers, the weight ratio of glutamic acid:glutamine from the free amino acids is greater than 1:1, in particular greater than 5:1 and very particularly greater than 25:1.

Here a glutamate precursor is meant to include glutamic acid or alfa-keto glutaric acid, a biochemical precursor. Glutamate can be in the form of a physiologically acceptable glutamate salt (for example the sodium, potassium, calcium or magnesium salt). As a source of glutamate protein hydrolysates can be used or freeze dried cultures of (lactic acid) bacteria (probiotics) which contain glutamate as a protecting agent. An example of such a lactic acid culture is Lactobacillus Reuteri, obtainable from Biogaia, originating from human milk.

Free glutamic acid is understood to be glutamic acid or a salt thereof that is not bound in protein or peptide and that has either been added or is present in the free amino acid fraction of a protein or hydrolysed protein (hydrolysed proteins usually contain 10-20% free amino acids) or is present as a protecting agent in a probiotic lactic acid culture.

The preparations of the invention are preferably combined with suitable prebiotics and probiotics, which have a beneficial effect on the intestinal flora. The prebiotics comprise short or long chain oligosaccharides, in particular galacto-oligosaccharides and fructo-oligosacclarides, branched oligosaccharides, sialyloligosaccharide, nucleotides, protein hydrolysates, sialic acid rich milk products or derivatives thereof, etc.

The nutritional preparation to be prepared according to the invention can be used in the treatment of all conditions where hyperpermeability of the intestinal wall is concerned. Examples of these are food allergy, allergy to internal drugs, sepsis and similar clinical conditions, translocation of pathogenic bacteria through the intestinal wall, endotoxaemia, viral diarrhoea, low intestinal blood flow, IC patients, patients after surgical interventions or with major burns, parenteral nutrition and undernutrition. It can also be used in the case of intestinal maturation of newborn babies, reduction of abnormal crying in children or the treatment of hyperactivity (Attention Deficit Hyperactivity Disorder, ADHD)

### EXAMPLE 1

The macromolecular permeability of the intestinal epithelium is controlled by the passage through intercellular tight junctions in the paracellular channels. Opening of these tight junctions is controlled by the epithelial cells in response to various intercellular mediators, such as Ca, cyclic AMP, G proteins and protein kinase c. The human intestinal cell line HT-29CL.19A is becoming increasingly more important for studying this paracellular permeability in vitro. See also the abovementioned article in J. Parenteral Enteral Nutrition, that is incorporated herein by reference.

For the present examples, confluent monolayers of HT-29CL19A cells were cultured on permeability filters. After 14-17 days the cells were allowed to grow for a further two days without glutamine in the medium. The transepithelial permeability from apical to basolateral was determined for horseradish peroxidase (HRP) with the aid of an enzyme assay. Phorbol 12,13-dibutyrate (PDB, 1 mmol/l) was used to increase the permeability. The effect of glutamine, glutamate and the g-glutamyl transferase inhibitor acivicin was investigated. All agents were added to the apical compartment.

It was found that PDB increases the HRP flux 3-fold compared with the control after 150-279 min stimulation (p<0.001). Glutamine reduces this hyperpermeability appreciably. Glutamate (0.6 mmol/l) had the same effect (p<0.001). Acivicin prevented the glutamine-mediated reduction in the hyperpermeability induced by PDB. This effect did not occur in the presence of glutamate.

It can be seen from this experiment that glutamate reduces the macromolecular hyperpermeability in HT-29CL19A cells.

### EXAMPLE 2

A complete, pulverulent, glutamic acid-containing baby food for premature children was prepared which had the following composition per 100 g powder-dry matter:

| desalinated whey, solids | 39.2 g |
|---|---|
| vegetable fats | 26.4 g |
| lactose | 17.9 g |
| skimmed milk, solids | 13.4 g |
| glucose syrup | 0.90 g |
| soy lecithin | 0.16 g |
| glutamic acid | 0.5 g |
| L-arginine | 0.05 g |
| taurine | 0.04 g |
| L-Tryptophane | 0.02 g |
| nucleotides | 0.03 g |
| microminerals and vitamins | 1.4 g |
| casein/whey protein ratio | 40/60 |
| % crude protein | 10.8 |
| % free glutamic acid | 0.5 |

A fluid composition that contains approximately 15% solids can be prepared from such a powder. Approximately 175 ml of the fluid composition is administered per kg body weight per day.

### EXAMPLE 3

A food was prepared as in example 2, with the exception that instead of 0.7 g lactose 0.7 g galacto-oligosaccharides were incorporated per 100 g powder.

### EXAMPLE 4

A complete, pulverulent, glutamic acid-containing baby food for children with an allergy was prepared which had the following composition per 100 g powder-dry matter:

| hydrolysed casein | 13.5 g |
|---|---|
| vegetable fat | 27 g |
| glucose syrup | 58.05 g |
| taurine | 0.04 g |
| L-carnitine | 0.01 g |
| microminerals and vitamins | 1.4 g |
| % crude protein | 12 |
| % free omithin | 0.01 |
| % free glutamic acid | 0.3 |

### EXAMPLE 5

A food was prepared according to example 4, with the exception that 0.25 g arginin and 0.005 g spermine and spermidine was incorporated instead of 0.255 g glucose syrup.

### EXAMPLE 6

A pulverent food for young children was prepared to limit excessive crying which contained per 100 gram:

| lactic protein | 11 g |
|---|---|
| fat | 27 g |
| lactose | 56 g |
| nucleotides | 0.03 g |
| glutamic acid | 0.45 g |
| minerals, vitamins, probiotic | 3.02 g |
| water | 2.5 g |
| ratio whey protein casein casein hydrolysate 40:30:30 | |
| L. Reuteri (Biogaja) | 1 × 10⁸ |
| % free glutamic acid | 0.5 |

### EXAMPLE 7

A pulverent food for older children with multiple functional properties was prepared, containing per 100 grams:

| lactic protein | 22.1 g |
|---|---|
| fat | 18.4 g |
| lactose | 39 g |
| sucrose | 10 g |
| alfa-ketoglutarate | 0.1 g |
| fructo-oligosaccharide (inulin) | 3 g |
| nucleotides | 0.05 g |
| minerals, vitamins, probiotics | 4.85 g |
| water | 2.5 g |
| L. Reuteri (Biogaia) | 1 × 10 ⁸ |
| casein/whey protein ratio | 75:25 |
| goat's milk protein (comprising human-milk like | |
| sialyloligosaceharides): 20% of | |
| the lactic protein | |
| % alfa-ketoglutarate | 0.1 |
| % free glutamic acid | 0.05 |
| % free glutarnic acid equivalents | 0.15 |

### EXAMPLE 8

A pulverent food for children having hyperactivity syndrome (ADHD) was prepared containing, compared to example 7, a casein/casein hydrolysate ratio in the lactic protein fraction of 40:60,

The product contains per 100 g

| L. Reuteri | 1 × 10 ⁸ |
|---|---|
| % free glutamic acid equivalents | 0.45 |

### EXAMPLE 9

A problem-solving, fluid, glutamic acid-containing enteral food based on caseinate and glutamine-rich vegetable hydrolysed protein (30% glutamine) was prepared which had the following composition per 100 g:

| | |
|---|---|
| caseinate | 5.2 g |
| glutamine-rich hydrolysed protein | 1.0 g |
| glutamic acid | 0.6 g |
| arginine | 0.2 g |
| fats | 3.4 g |
| carbohydrates | 9.5 g |
| minerals and vitamins | 0.4 g |
| lecithin | 0.1 g |
| water | 79.6 g |
| % crude protein | 6.3 g |
| % free arginin | 0.2 g |
| % glutamin | 0.3 g |
| % free glutamic acid | 0.6 g |

1. Use of glutamate and/or a glutamate precursor for the preparation of a nutritional or pharmaceutical preparation for the treatment or prevention of hyperpermeability or undesired permeability of the intestinal wall.

2. Use according to claim 1, wherein the nutritional preparation is an infant formula or children's food.

3. Use according to claim 1 or 2, wherein the nutritional preparation is an enteral food or a food supplement.

4. Use according to any one of claims 1 to 3, wherein the nutritional preparation also contains lactic proteins or hydrolysed lactic proteins.

5. Use according to any one of claims 1 to 4, wherein the nutritional preparation also contains vegetable proteins or hydrolysed products thereof.

6. Use according to any one of claims 1 to 5, wherein hydrolysed protein is used as the source of glutamate.

7. Use according to any one of claims 1 to 6, wherein the glutamate precursor is glutamic acid or alfa-keto glutaric acid.

8. Use according to any one of the preceding claims, wherein the nutritional preparation also contains glutamine or an equivalent thereof.

9. Use according to claim 8, wherein the weight ratio of glutamic acid: glutamine in the free amino acid fraction is greater than 1:1, in particular greater than 5:1 and preferentially greater than 25:1.

10. Use according to any one of the preceding claims, wherein the preparation further contains one or more polyamines in particular spermine, spermidine or putrescine and/or one or more polyamine precursors, in particular ornithin and arginin.

11. Use according to any one of the preceding claims, wherein the nutritional preparation contains 0.05-10 g, preferably 0.2-4 g free glutamate per 100 g nutritional preparation (dry weight).

12. Use according to any one of the preceding claims, wherein the nutritional preparation further contains one or more prebiotics, preferably selected from the group consisting of protein hydrolysates, nucleotides, galacto-oligosaccharides, fructo-oligosaccharides, branched oligosaccharides and sialyloligosaccharides or equivalents thereof.

13. Use according to any one of the preceding claims, wherein the nutritional preparation contains freeze dried probiotics, in particular freeze dried Lactobacillus Reuteri as the source of glutamate.

14. Use according to any one of the preceding claims, in the case of deterioration of the mucosal barrier, intestinal dysfunction or injury, suboptimal intestinal wall maturation of new-borns, undernutrition, suboptimal intestinal blood flow, allergy, sepsis, translocation of pathogenic bacteria through the intestinal wall, endotoxaemia, viral diarrhoea, regularly crying children, hyperactive children, IC patients, patients after surgery or major burns.

15. Preparation containing free glutamate and/or a glutamate precursor and polyamines, in particular spermine, spermidine or putrescine and/or one or more polyamine precursors, in particular ornithin and arginin.
